(19) United States
(12) Patent Application Publication
(10) Pub. No.: US 2001/0019839 A1
(43) Pub. Date: Sep. 6, 2001

(54) Method for production of a C1 esterase inhibitor (C1-INH)-containing composition

(76) Inventors: Wolfgang Schoenhofer, St. Poelten (AT); Hans-Peter Schwarz, Vienna (AT); Oliver Zoechling, Vienna (AT); Yendra Linnau, Vienna (AT)

Correspondence Address: Baxter Healthcare Corporation, P.O. Box 15210, Irvine, CA, 92614, US

(21) Appl. No.: 09/746,625

(22) Filed: Dec. 21, 2000

(30) Foreign Application Priority Data
Dec. 22, 1999 (AT) A2166/99

(51) Int. Cl. C12N 9/99; A61K 38/55

(52) U.S. Cl. 435/184; 514/12

(57) ABSTRACT

A method is described for production of a C1-INH esterase inhibitor (C1-INH)-containing composition, which includes the following steps:

treating a C1-INH-containing starting material with an anion exchanger under acidic conditions and

eluting the C1-INH from the anion exchanger, in which a C1-INH-containing composition is obtained.

### FIELD OF THE INVENTION

The invention concerns a method for production of a C1 esterase inhibitor (C1-INH)-containing composition, as well as improved compositions containing C1-INH and C1-INH-containing combination preparations.

### BACKGROUND OF THE INVENTION

C1-INH is a plasma protease inhibitor which plays a central role in regulating the activation of complement and the kinin generation system. C1-INH is the only inhibitor of C1r and C1s in plasma, and is responsible for roughly half the kallikrein-activating activity and most of the blood coagulation factor XII inactivation. C1-INH also inhibits blood coagulation factor XIa.

C1-INH consists of a single polypeptide chain with 478 amino acids and is synthesized with a 22 amino acid signal sequence. Based on sequence homology to the serpins, C1-INH has been assigned to the serpin “superfamily” of serine protease inhibitors.

In contrast to other proteases, especially from this family, or other proteins in blood plasma, C1-INH has an extremely high degree of glycosylation. About 50% of the total weight of C1-INH (about 105 kd) is composed of carbohydrates; the molecular weight of the peptide chain is approx. 53 kd.

The isoelectric point of C1-INH lies near 2.7 to 2.8 in the α₂ electrophoretic mobility determination.

C1-INH can be produced for example, from human plasma or by using recombinant techniques. It was found that C1-INH variants with nonphysiological glycosylation patterns (perhaps without N-glycosylation; by expression in hepatoma cell lines in the presence of tunicamycin) retain inhibitory activity, especially against C1s. Amino-terminally truncated C1-INH molecules also exhibit unaltered activity relative to C1s, even though the main portion of the glycosylation sites lie in the amino terminal region (see Davis “Structure and Function of C1 Inhibitor,” Behring Inst. Mitt., 84 (1989), 142-150).

C1-INH is used in human medicine mostly because of its known inhibitory activity in the complement system. Thus, C1-INH can moderate undesired pharmacological side effects. The addition of C1-INH is therefore useful when applying protein preparations, which can exhibit side effects because of undesired pharmacologically active substances, in order to moderate the side effects. In this case, C1-INH can be administered right before administration of the potentially side-effect-burdened preparation to the patient or in combination with the active principle being administered from biological sources, especially with plasma proteins or plasma derivatives (EP-0 119 990 B1).

Another important area of application of C1-INH is the treatment of hereditary or acquired angioedemas. Hereditary angioedema (HAE) is a rare, autosomal-dominant inheritable gynecotropic disease, which is characterized by a C1-INH deficiency or by formation of defective C1-INH. Acute attacks triggered by stressful situations occur frequently in HAE patients, with edematous swelling in the skin (mostly on the face and extremities) and mucosa. Serious abdominal colic can occur in edemas of the gastrointestinal mucosa, often connected with vomiting and diarrhea.

The greatest hazard in HAE, however, results from attacks to the upper respiratory tract. Life-threatening asphyxiation attacks can occur in such laryngeal edemas. The high mortality of HAE (about 20 to 30%) essentially is attributed to the occurrence of such laryngeal edemas.

HAE is mostly treated with C1-INH, in addition to treatment with adrenalin, cortisone, danazol and ε-aminocaproic acid (see Mohr et al., Anaesthesist 45 (1996), 626-630, as well as Davis, Immunodeficiency Reviews 1 (1989), 207-226).

In cases where acquired angioedemas are treated with C1-INH, mostly those angioedemas occurring from C1-INH deficiency in the scope of tumors or autoimmune diseases are relevant (see Pschyrembel, “Klinisches Wörterbuch,” 257^{th} Edition, page 71).

A number of methods have been proposed to produce C1-INH-containing compositions from plasma, including, among others, affinity chromatography, ion exchange chromatography, gel filtration, precipitation, and hydrophobic interaction chromatography. It has been found, however, that C1-INH often cannot be adequately separated from its direct accompanying proteins with these methods (EP-0 101 935 B1). Combinations of specific purification steps were therefore increasingly proposed in the prior art.

A C1-INH production method is described in EP-0 101 935 B1, in which a C1-INH-containing starting material is processed by a combination of precipitation steps and hydrophobic chromatography to produce a C1-INH preparation, which was about 90% pure at a yield of about 20%.

A combination of PEG precipitation and chromatography over jacalin-agarose and hydrophobic chromatography is proposed in U.S. Pat. No. 5,030,578 A. A combination of ion exchange chromatography on DEAE groups, affinity chromatography using immobilized heparin, and treatment with a strong cation exchange gel was further proposed by Poulle et al. (Blood Coagulation and Fibrinolysis 5 (1994), 543-549; U.S. Pat. No. 5,681,750 A). The C1-INH preparation obtained with this method exhibits a specific activity of 6.5±0.5 units/mg, but an antigen/activity ratio of only 1.7 to 2. In plasma, the antigen/activity ratio of 1:1.

It was found according to the methods as described in the prior art, that either the accompanying proteins could not be separated efficiently enough from C1-INH (mostly only albumin is insufficiently separable from C1-INH with the described methods) or that satisfactory separation of these accompanying proteins at the expense of C1-INH activity must be accepted, leading to an unsatisfactory specific activity or antigen/activity ratio in the obtained C1-INH preparation. The provision of numerous chromatographic steps is also a shortcoming for the yield and activity of the obtained preparation, since both a loss of yield and a loss of activity must be tolerated with each chromatography step, for example, because of denaturation.

### SUMMARY OF THE INVENTION

The task of the present invention is therefore to prepare an improved method for production of a C1-INH-containing composition, which permits simple and efficient separation of C1-INH-accompanying proteins, especially albumin, is applicable on an industrial scale, and can lead to improved C1-INH preparations in combination with already known process steps.

This task is solved according to the invention by a method for production of a C1-INH-containing composition that includes the following steps:

treating a C1-INH-containing starting material with an anion exchanger under acidic conditions, in which C1-INH is bonded to the anion exchanger, and

treating C1-INH from the anion exchanger, thereby determining a C1-INH-containing composition.

The present invention is based on the surprising finding that treatment of C1-INH-containing material with anion exchangers at an acid pH (i.e., below pH 7) leads to efficient separation of undesired accompanying proteins. Anion exchanger treatment has indeed long been known as a means of C1-INH purification, but thus far adsorption of C1-INH on an anion exchanger under acidic conditions has never been attempted. This circumstance is attributed to the fact that usual treatment with anion exchangers (not only for C1-INH) is conducted at neutral or basic pH, since it is only in these ranges that anion exchange capacity is considered sufficient, primarily in purification methods on an industrial scale.

However, it was found according to the invention that, precisely under acidic conditions, the bonding of C1-INH to the anion exchanger functions efficiently, and undesired accompanying proteins are not bonded and can be depleted. This is also surprisingly true for proteins, for example albumin, which like C1-INH, have a low pI value. It turned out, surprisingly, that anion exchange chromatography can efficiently separate these proteins from C1-INH, even at a pH that lies above the pI value of the proteins being eliminated.

The C1-INH-containing starting material is preferably treated with the anion exchanger at a pH value of 3.0 to 6.9, preferably pH 4.5 to 6. At pH values of 7.0 and higher, the effects according to the invention, especially efficient separation of accompanying proteins with low pI values, no longer occur satisfactorily. At pH values less than 3.0, the invention can be performed in principle, but the risk of denaturation losses of acid-labile proteins or other materials used during purification must then be tolerated. An ionic strength of 30 mS (0.5 M NaCl) or higher is preferably used during adsorption.

Since the C1-INH preparation obtained with the present invention is to be used mostly pharmaceutically, at least one additional step for inactivation of potentially present viruses is provided in the method according to the invention. This can occur before, during, or after the anion exchange step. Appropriate virus inactivation steps are generally known. They include chemical, chemical-physical, and physical methods. Methods using virucidal substances can also be employed during and after a chromatographic purification method.

At least two measures are preferably provided that cause inactivation or depletion of human pathogenic infection producers, including viruses transmittable by blood, like HIV, HAV, HBV, HCV, HGV and parvo viruses, but also the infectious pathogens of BSE and CJD.

Effective measures for inactivation of viruses include, for example, treatment with organic solvents and/or detergents (EP-0 131 740 A, EP-0 050 061 A, WO98/44941 A), treatment with chaotropic agents (WO90/15613 A), heat treatment methods, preferably in the lyophilized, dry, or moist state EP-0 159 311 A), combination methods (EP-0 519 901 A), and physical methods. The latter cause viral inactivation, for example, by irradiation with light, perhaps in the presence of photosensitizers (EP-0 471 794 A and WO-97/3768 A).

Depletion methods for human pathogens using ultrafilters, low-pass filters, and especially nanofilters, are particularly preferred according to the invention (WO97/40861 A, 4998/57672 A), but precipitation steps and other protein purification measures, like adsorption, also contribute, in principle, to depletion of any pathogens that might be present.

The nanofiltration particularly preferred according to the invention is preferably conducted so that the C1-INH-containing composition is diluted before the nanofiltration step. Problems that can occur from the relatively high molecular weight of C1-INH, and can lead, for example, to clogging of the filter pores, are avoided from the outset on this account. Nanofiltration is preferably conducted within the scope of the method according to the invention after anion exchange chromatography, and preferably with filters that have a pore size from 10 to 40 nm.

Any C1-INH-containing material is suitable in principle as C1-INH-containing material. However, plasma, cryosupernatant, C1-INH-containing Cohn fractions, C1-INH-containing cell culture supernatants, transgenically produced C1-INH-containing material, or a prepurified C1-INH preparation are preferably used. The prepurified C1-INH preparation can then be obtained by a method already described in the prior art before it is subjected, according to the invention, to the anion exchange step under acidic conditions.

Even further improved purification results can be achieved according to the invention by repeating the anion exchange step under acidic conditions. The pH of the obtained solution is then optimally brought to an acid value again, but then brought in contact with an anion exchanger, in which C1-INH is bonded again. As in the first anion exchange treatment, the adsorbed C1-INH can also be subjected to one or more washing steps before being eluted again from the anion exchanger.

The C1-INH-containing composition obtained after elution can, in addition to the preferred retreatment with the anion exchange step according to the invention, also be purified further using other methods. The additional purification steps preferred according to the invention include those steps whose essential effectiveness has already been described in the prior art with respect to C1-INH, like precipitation (with PEG, ammonium sulfate, etc.), hydrophobic chromatography, especially over phenylsepharose, affinity chromatography, especially over heparin sepharose or jacalin-agarose, or cation exchange chromatography.

All anion exchangers that have an affinity to C1-INH can be considered as anion exchangers in principle, like anion exchangers based on cellulose (Whatman® DE52, QAE52, Express Ion®Q and D, all from the Whatman company) with diethylaminoethyl groups (DEAE-Sephacel®), anion exchangers based on crosslinked dextran with diethylaminoethyl groups (DEAE-Sephadex®), anion exchangers based on agarose with diethylaminoethyl groups (DEAE-Sepharose CL6B®, DEAE-Sepharose Fast Flow®), anion exchangers based on crosslinked dextran with diethyl[2-hydroxypropyl]aminoethyl groups (QAE-Sephadex®), anion exchangers based on agarose with CH₂N⁺(CH₃)₃ groups (Q-Sepharose Fast Flow®, Q-Sepharose High Performance®, Q-Sepharose Big Beads®) (all from Pharmacia), spherical chromatography gels produced by copolymerization of N-acryloyl-2-amino-2-hydroxymethyl-1,3-propanediol and an anionic acrylic derivative with diethylaminoethyl groups as functional anion exchangers (DEAE-Tris-Acryl®), noncompressible silica-dextran matrices, in which porous silica gel is embedded in a crosslinked dextran matrix, with reactive diethylaminoethyl anion exchanger groups (DEAE-Spherodex®), gels from rigid polystyrene particles, whose pores are filled with a hydrogel carrying quaternary amino groups with strong anion exchange effects (Q-Hyer-D®) (all from Sepracor); rigid macroporous hydrophilic surfaces with N⁺(C₂H₅)₂ or N⁺(CH₃)₃ groups (Macroprep DEAE®, Macroprep Q®) (all from BioRad); anion exchangers with diethylamino-diethyl(2-hydroxypropyl)aminoethyl and CH₂N⁺(CH₃)₃ groups (DEAE-Toyopearl®, QAE-Toyopearl®, Toyopearl SuperQ®) (all from Tosohaas); anion exchange resins, consisting of porous polymethacrylate/polyacrylate gel (Protein PAK DEAE® from the Waters company); anion exchangers based on copolymers consisting of oligoethylene glycol dimethacrylate, glycidyl methacrylate and pentaerythritol dimethacrylate with a hydrophobic surface (Fractoge EMD-TMAE®, Fractogel EMD-DEAE®, Fractogel EMD-DMAE®), and anion exchangers based on silica with porous spherical pressure-stable chromatography particles (Licrospher 1000 TMAE®, Licrospher 1000 DEAE® and Licrospher 4000 DMAE®) (all from Merck).

According to the invention, anion exchanger materials, like DEAE-Sephadex®, QAE-Sephadex® A50 or Toyopearl Super-Q® 650C, as well as Whatman® DE52, QAE52, Express Ion®Q and D, are particularly preferred.

The purified C1-INH compositions obtained are preferably lyophilized and optionally subjected to (additional) virus-inactivation treatment. Heat treatment, especially in the temperature range between 60 and 100° C. over a period from 10 to 80 h, is preferred here according to the invention.

For use as pharmaceutical agents, the obtained C1-INH composition (lyophilized or in solution) is prepared to a pharmaceutical preparation and packed in the corresponding containers. Both stabilizers and other auxiliaries and/or other active components (to produce a combination preparation) can then be mixed with the C1-INH-containing composition, as according to EP-0 480 906 A, where lys-plasminogen is administered, combined with C1-INH.

A particularly preferred variant of the method according to the invention is characterized by the sequence of the following steps:

treating a C1-INH-containing starting material with an anion exchanger under acidic conditions, in which C1-INH is bonded to the anion exchanger,

optional washing of the adsorbed C1-INH-containing material,

eluting the C1-INH from the anion exchanger, in which a C1-INH-containing eluate is recovered,

treating the C1-INH-containing eluate with PEG, preferably with PEG 4000, especially in amounts of less than 15%, in which a precipitate and a C1-INH-containing supernatant are obtained,

treating the C1-INH-containing supernatant with a detergent, in which any viruses present are inactivated,

treating the detergent-containing C1-INH-containing supernatant with an anion exchanger, in which C1-INH is bonded again and the detergent and any contaminant still present are removed,

optional washing of the bonded C1-INH-containing material,

eluting the C1-INH from the anion exchanger, in which a virus-inactivated C1-INH-containing eluate is obtained,

nanofiltrating of this eluate,

lyophilizing of the nanofiltered C1-INH solution, and

heat treating of the lyophilized C1-INH-containing composition.

Elution from the anion exchanger preferably occurs with a buffer having a salt concentration higher than the salt concentration in the adsorption step, the best results being achieved with salt concentrations that lie at least 3 times higher than that of the adsorption solution.

The washing step of the adsorbed C1-INH is preferably conducted with the adsorption buffer, or a buffer that corresponds roughly to the adsorption buffer, especially in terms of conductivity. The salt concentration of the washing buffer preferably lies no more than 10 to 100% above that of the adsorption solution.

According to another aspect, the present invention concerns C1-INH-containing compositions characterized by the fact that they have a specific activity of 2.0 units/mg of protein or more at an antigen/activity ratio of less than 1.5. As also demonstrated in the examples, the method according to the invention can lead to highly purified preparations in this way. In the prior art, C1-INH-containing compositions have already been obtained with a specific activity of higher than 2 units/mg of protein or with an antigen/activity ratio of less than 1.5, but the combination of this degree of purification could never previously be achieved, since as already described, the increased specific activity always occurred at the expense of the antigen/activity ratio, or an improved antigen/activity ratio could never be achieved with such high specific activities.

Compositions with a specific activity of 4 to 8, especially 5 to 7, units/mg of protein are attainable without difficulty according to the invention. Antigen/activity ratios from 1 to 1.4, especially 1.1 to 1.3, are attainable simultaneously.

The preparations according to the invention are preferably present as pharmaceutical preparations in packaged form and are optionally virus-inactivated.

According to another aspect, the present invention concerns combination preparations that include a C1-INH-containing composition according to the invention with at least one additional pharmaceutically active substance (similar to the drugs described in EP-0 119 990 B1 and EP-0 480 906 A).

The invention is further explained by means of the following example:

### EXAMPLE

2.5 g of dry QAE-Sephadex A50® is equilibrated with a C1-INH solution (1000 IU C1-INH (one international unit (IU) or unit (U) of C1-INH corresponds to the C1-INH activity in 1 mL of fresh plasma), 100 mM sodium acetate, 50 mM sodium chloride, pH 5.5) and the pH set at 5.5. Adsorption is carried out for 2 h at 4° C.

The gel with the adsorbed C1-INH is then washed with:

a) 100 mM sodium acetate and 50 mM sodium chloride, pH 5.5, and

b) 20 mM Tris and 200 mM sodium chloride, pH 7.5.

It is eluted with 20 mM Tris and 750 mM sodium chloride, pH 7.5.

The obtained C1-INH solution is brought back to a pH of 5.5, and PEG 4000 is added to a final concentration of 12% (w/w). It is precipitated for 1 h at 4° C. and then centrifuged, in which the precipitate is discarded.

b 12.5% Tween 80® (w/w) is added to the supernatant and agitated for 4 h at 35° C.

This Tween 80®-containing solution or suspension is equilibrated with 10 mM sodium acetate and 50 mM sodium chloride, pH 5.5, in which about 20 IU C1-INH per mL of gel is adsorbed. The adsorbed gel is then washed with

a) 10 mM sodium acetate and 50 mM sodium chloride, pH 5.5,

b) 154 mM NaPO₄ buffer at pH 5.5, and

c) 10 mM Tris and 100 mM sodium chloride at pH 7.0.

Elution is conducted with a solution containing 10 mM Tris and 250 mM sodium chloride at pH 7.0.

The obtained eluate is nanofiltered with an Asahi Planova 15 N filter; the nanofiltered solution is then ultra/diafiltered.

The obtained solution is standardized at the desired concentration (50, 100 or 200 international units per mL).

1 g/L sodium citrate, 1 g/L trehalose, and 9 g/L sodium chloride are provided in the buffer. This preparation is lyophilized to a moisture content of less than 1.5% and heated in the final containers to at least 80° C. for at least 72 h.

The results are shown in the following table:

TABLE

| | | | Specific activity |
|---|---|---|---|
| | Yield/step | | IU C1-INH/ |
| Sample | Average | (Example) | mg of protein |
| Starting material | 100% | (100%) | 0.02 |
| Eluate 1^{st} anion exchange | 70-115% | (71%) | 1.1 |
| PEG supernatant | 75-115% | (88%) | 1.8 |
| After Tween 80 | 75-115% | (110%) | 1.8 |
| Eluate 2^{nd} anion exchange | 75-115% | (100%) | 5.2 |
| After 15 nm nanofiltration | 75-115% | (85%) | 6.0 |
| After lyophilization | 75-115% | (79%) | 6.0 |
| After heat treatment | 75-115% | (110%) | 6.0 |

The end product so obtained has an antigen/activity ratio of 1.15:1.

1. A method for production of a C1-INH esterase inhibitor (C1-INH)-containing composition, including the following steps:
treating a C1-INH-containing starting material with an anion exchanger under acidic conditions and
eluting the C1-INH from the anion exchanger, in which a C1-INH-containing composition is obtained.

2. The method according to claim 1, wherein the treatment of the starting material with the anion exchanger is conducted at a pH from 3.0 to 6.9.

3. The method according to claim 2, wherein the anion exchange is contacted at a pH from 4.5 to 6.

4. The method according to claim 1, wherein at least one additional step for inactivation of potentially present viruses is conducted.

5. The method according to claim 1, wherein plasma, cryosupernatant, C1-INH-containing Cohn fractions, C1-INH-containing cell culture supernatants, transgenically produced C1-INH-containing material, or a prepurified C1-INH preparation is used as starting material.

6. The method according to claim 1, wherein the composition obtained after elution is brought in contact with an anion exchanger again, in which the C1-INH is bonded, the bonded C1-INH is optionally subjected to at least one washing step, and eluted again.

7. The method according to claim 1, wherein the composition obtained after elution is subjected to at least one additional purification step.

8. The method according to claim 7, wherein the purification step is PEG precipitation, hydrophobic chromatography, affinity chromatography or cation exchange chromatography.

9. The method according to claim 1 additionally comprising the step of lyophilizing said C1-INH-containing composition.

10. The method according to claim 9, wherein the lyophilized composition is subjected to heat treatment.

11. The method according to claim 10, wherein the heat treatment is in a temperature range between 60° C. and 100° C. over a period from 10 to 80 h.

12. The method according to claim 1, wherein the obtained composition is prepared as a pharmaceutical preparation.

13. The method for production of a C1-INH-containing composition comprising the following steps:
treating a C1-INH-containing starting material with an anion exchanger under acidic conditions, in which C1-INH is bonded to the anion exchanger,
eluting the C1-INH from the anion exchanger, in which a C1-INH-containing eluate is recovered,
treating the C1-INH-containing eluate with PEG, especially in an amount of less than 15%, in which a precipitate and a C1-INH-containing supernatant are obtained,
treating the C1-INH-containing supernatant with a detergent, in which any viruses present are inactivated,
treating the detergent-containing C1-INH-containing supernatant with an anion exchanger, in which C1-INH is bonded again, and the detergent and any contaminants still present are removed,
optional washing of the bonded C1-INH-containing material,
eluting of the C1-INH from the anion exchanger, in which a virus-inactivated C1-INH-containing eluate is obtained,
nanofiltrating of this eluate,
lyophilizing the nanofiltered C1-INH solution, and
heat treating of the lyophilized C1-INH-containing composition.

14. The method according to claim 13, wherein the C1-INH containing material adsorbed to the anion exchanger is washed.

15. A C1-INH-containing composition having a specific activity of 2.0 U/mg of protein or more at an antigen/activity ratio of less than 1.5.

16. The C1-INH-containing composition according to claim 15 having a specific activity of 4 to 8 U/mg of protein.

17. The C1-INH containing composition according to claim 15 having a specific activity of 5 to 7 U/mg of protein.

18. The C1-INH-containing composition according to claim 15 having an antigen/activity ratio of 1 to 1.4.

19. The C1-INH containing composition according to claim 15 having an antigen/activity ratio of 1.1 to 1.3.

20. The C1-INH-containing composition obtained by the method of claim 1, wherein said composition is present as a pharmaceutical preparation.

21. A C1-INH-containing composition obtained by the method of claim 4.

22. A combination preparation according to claim 15, and at least one additional pharmaceutically active substance, selected from the group consisting of plasma protein or plasma derivative.
